(19) United States
(12) Patent Application Publication
(10) Pub. No.: US 2026/0294301 A1
(43) Pub. Date: Oct. 1, 2026

(54) EMOTION ESTIMATION APPARATUS AND EMOTION ESTIMATION METHOD

(71) Applicants: HONDA MOTOR CO., LTD., Tokyo (JP); VIRGINIA TECH INTELLECTUAL PROPERTIES, INC., Blacksburg, VA (US)

(72) Inventors: Aya Sasaki, Wako-shi (JP); Tomoaki Ohashi, Wako-shi (JP); Nariaki Kuriyama, Wako-shi (JP); Feng Guo, Blacksburg, VA (US); Shu Han, Blacksburg, VA (US); Boyu Jiang, Blacksburg, VA (US); Manhua Wang, Blacksburg, VA (US); Liang Shi, Blacksburg, VA (US)

(21) Appl. No.: 19/090,592

(22) Filed: Mar. 26, 2025

(51) Int. Cl. A61B 5/16; A61B 5/00; A61B 5/18

(52) U.S. Cl. CPC A61B 5/165; A61B 5/18; A61B 5/6893; A61B 2560/0242

(57) ABSTRACT

An emotion estimation apparatus includes a biological information detection unit that detects biological information of a driver of a target vehicle, an environment detection unit that detects environment information of a surrounding area of the target vehicle, and an emotion estimation unit that estimates a strong emotion of the driver based on the biological information and the environment information.

### BACKGROUND OF THE INVENTION

### Field of the Invention

The present invention relates to an emotion estimation apparatus and an emotion estimation method.

### Description of the Related Art

An information processing apparatus has been proposed that predicts a driving risk based on biological data of a driver, behavior of a vehicle body, and the like (see, for example, International Publication No. 2018/190152).

International Publication No. 2018/190152 also describes estimating, based on biological data of a user of interest, a degree of emotion of the user of interest, for example, a degree of excitement, a degree of restlessness, and a degree of anxiety.

Meanwhile, International Publication No. 2018/190152 is unclear about a specific method for processing of estimating the degree of emotion based on the biological data.

It is desirable to quickly and appropriately estimate an emotion influencing driving error behavior of the driver for this type of technique.

In order to solve the above-described problem, an object of the present application is to facilitate quick and appropriate estimation of the emotion influencing the driving error behavior of the driver.

### SUMMARY OF THE INVENTION

As an aspect according to the present disclosure, an emotion estimation apparatus includes a biological information detection unit that detects biological information of a driver of a target vehicle, an environment detection unit that detects environment information of a surrounding area of the target vehicle, and an emotion estimation unit that estimates a strong emotion of the driver based on the biological information and the environment information.

As another aspect according to the present disclosure, an emotion estimation method executed by a computer includes a biological information detection step of detecting biological information of a driver of a target vehicle, an environment detection step of detecting environment information of a surrounding area of the target vehicle, and an emotion estimation step of estimating a strong emotion of the driver based on the biological information and the environment information.

According to the present invention, it is possible to facilitate quick and appropriate estimation of an emotion influencing driving error behavior of a driver.

### BRIEF DESCRIPTION OF THE DRAWINGS

FIG. 1 is a diagram showing an overview of a vehicle control apparatus according to the present embodiment;

FIG. 2 is a diagram showing a relationship between strong emotions and a driving risk;

FIG. 3 is a diagram showing the vehicle control apparatus and a peripheral configuration thereof;

FIG. 4 is a flowchart showing an operation of the vehicle control apparatus;

FIG. 5 is a diagram for describing static traffic environment information;

FIG. 6 is a diagram for describing the static traffic environment information;

FIG. 7 is a diagram for describing a biology-emotion table;

FIG. 8 is a diagram for describing driving error behavior prediction; and

FIG. 9 is a diagram for describing driving risk prediction.

### DETAILED DESCRIPTION OF THE PREFERRED EMBODIMENTS

### Embodiment

### 1. Overview of Vehicle Control Apparatus 1

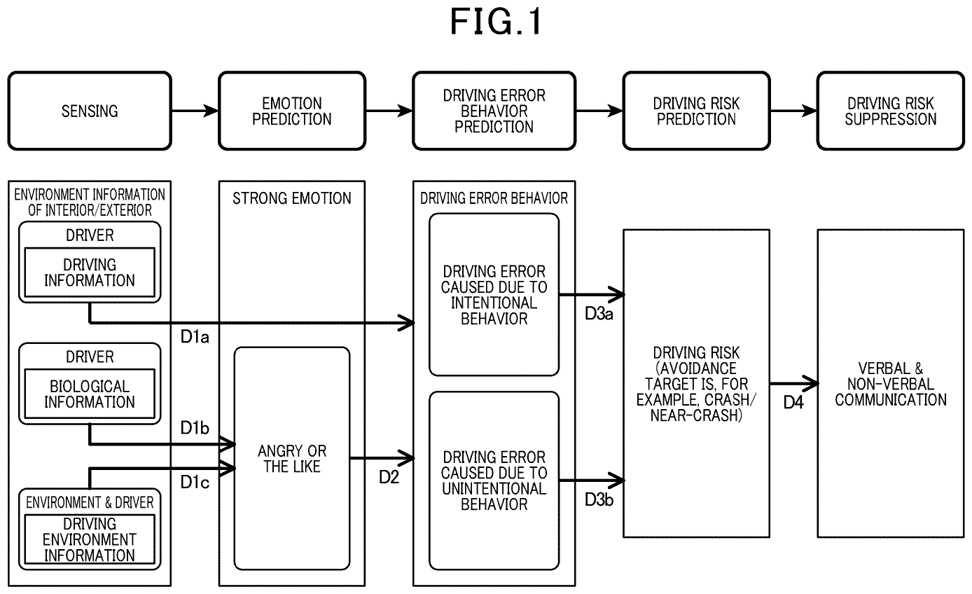

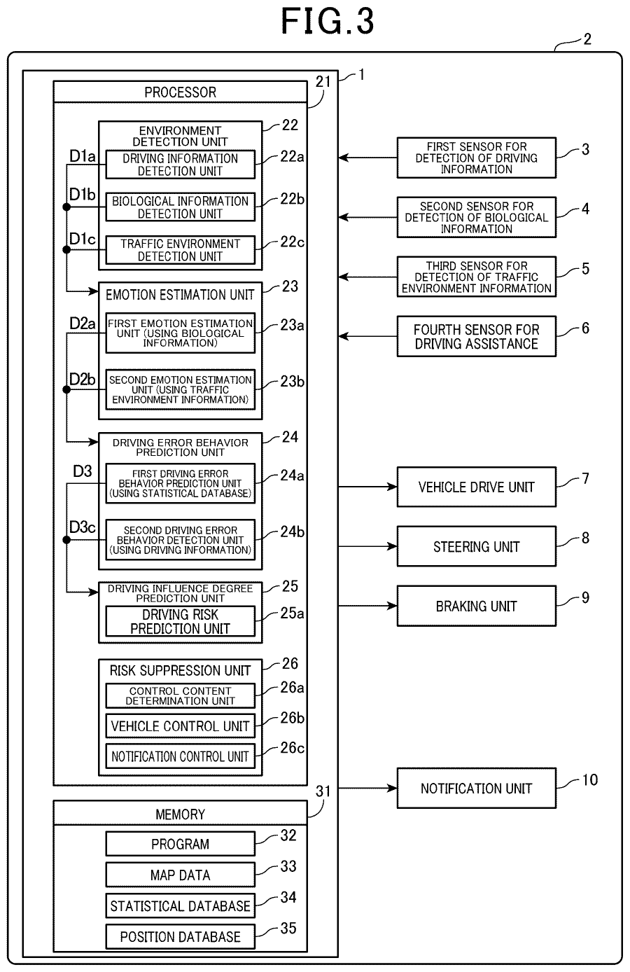

FIG. 1 is a diagram showing an overview of a vehicle control apparatus 1 according to the present embodiment. As shown in FIG. 1, the vehicle control apparatus 1 is an apparatus having functions for executing sensing processing S1 of detecting environment information D1 about at least one of an interior and an exterior of a target vehicle (a vehicle 2 shown in FIG. 3), emotion estimation processing S2 on a driver based on the environment information D1, driving error behavior prediction processing S3 based on the estimated emotion and the like, driving risk prediction processing S4 based on driving error behavior, and driving risk suppression processing S5.

The vehicle control apparatus 1 according to the present embodiment is an in-vehicle apparatus installed in the target vehicle, but is not limited to this configuration, and may also be an apparatus that remotely controls the target vehicle by communicating with the target vehicle. The target vehicle is not particularly limited, but a four-wheeled vehicle is described as an example thereof in the present embodiment.

### 1.1 Sensing Processing S1

Processing of detecting the environment information D1 about the interior and the exterior of the target vehicle is applied as the sensing processing S1. The environment information D1 is environment information that influences the driving, emotion, and the like of the driver. In the present embodiment, processing of detecting, as the environment information D1 about the interior and the exterior of the target vehicle, driving information D1a about the driver, biological information D1b about the driver, and traffic environment information D1c that allows identification of a traffic environment consisting of a surrounding environment is applied.

The driving information D1a is information that allows identification of the driving error behavior of the driver. To be specific, the driving information D1a is information relating to driving operations such as an accelerator operation, a brake operation, and a steering wheel operation, and corresponds to information obtained by directly or indirectly detecting each driving operation.

Examples of the information obtained by directly detecting each driving operation include an accelerator position, a brake operation amount, and a steering wheel operation amount. Examples of the information obtained by indirectly detecting each driving operation include vehicle dynamics information obtained by detecting movement of a front, back, left, right, and the like of the target vehicle.

The biological information D1b is biological information that influences the driving error behavior, the emotion, and the like of the driver. To be specific, the biological information D1b corresponds to information that allows identification of an expression, speech, and a gesture of the driver, or to a physiological signal that allows identification of a pulse, a blood pressure, and the like of the driver.

The traffic environment information D1c is information that allows identification of a traffic environment that influences the driving error behavior, the emotion, and the like of the driver. To be specific, the traffic environment information D1c corresponds to, for example, a region where the target vehicle is located, road conditions, how the driver grips the steering wheel, time information indicating the time of day such as daytime or nighttime, in-vehicle environment information indicating the presence or absence of passengers or the like, weather information indicating the weather and the like. The road conditions include the presence or absence of a road merge, the presence or absence of a construction zone, a state of road infrastructure, traffic regulation conditions, junction relationships, a slope, and the like.

### 1.2 Emotion Estimation Processing S2

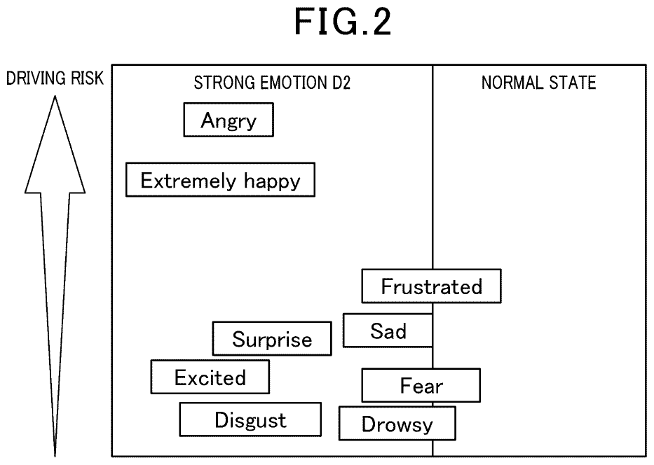

Processing of estimating an emotion including a strong emotion D2 of the driver by using the biological information D1b and the traffic environment information D1c is applied as the emotion estimation processing S2. The strong emotion D2 indicates a strong emotion that greatly influences a person's thoughts, behavior, and the like, and corresponds to, for example, “Angry”, “Extremely happy”, “Surprise”, and “Excited” shown in FIG. 2.

FIG. 2 is a diagram schematically showing a relationship between the strong emotion D2 and the driving risk.

The driving risk indicates the likelihood of a situation to be avoided occurring while driving. An example of the situation to be avoided includes trouble to be avoided while driving, and in the present embodiment, a crash event and a near-crash event are each set as the situation to be avoided.

The strong emotion D2 tends to greatly influence the driving error behavior as compared to when the driver is in a normal state, resulting in an increased driving risk. Especially certain emotions, including “angry” and “extremely happy” may further increase the driving risk as compared to other emotions.

### 1.3 Driving Error Behavior Prediction Processing S3

Processing of predicting future driving error behavior of the driver by using the estimated strong emotion D2 and the like is applied as the driving error behavior prediction processing S3. In the present embodiment, the driving error behavior prediction processing S3 includes processing of detecting real-time or past driving error behavior D3c included in the driving information D1a, in addition to the processing of predicting future driving error behavior D3a and D3b based on the estimated strong emotion D2.

Note that among the driving error behavior D3a and D3b, the driving error behavior D3a indicates driving error behavior caused due to intentional behavior of the driver. The driving error behavior D3b indicates driving error behavior caused due to unintentional behavior of the driver.

### 1.4 Driving Risk Prediction Processing S4

Processing of predicting the driving risk based on the predicted driving error behavior is applied as the driving risk prediction processing S4. In the present embodiment, an occurrence probability D4 of the crash event and the near-crash event is predicted as the driving risk.

### 1.5 Driving Risk Suppression Processing S5

Control for suppressing the predicted driving risk (the occurrence probability D4) is applied as the driving risk suppression processing S5. In the present embodiment, it is possible to reduce the driving risk and effectively prevent an unpredicted situation during driving, by performing predetermined control in accordance with the predicted driving risk.

### 2. Peripheral Configuration of Vehicle Control Apparatus 1

A peripheral configuration of the vehicle control apparatus 1 will be described with reference to FIG. 3.

The vehicle 2 includes a first sensor 3, a second sensor 4, a third sensor 5, a fourth sensor 6, a vehicle drive unit 7, a steering unit 8, a braking unit 9, and a notification unit 10, each of which are connected to the vehicle control apparatus 1.

The first sensor 3 is one or more sensors that detect information relating to the driving information D1a, and specifically correspond to an accelerator sensor, a brake sensor, steering wheel sensor, an acceleration sensor, a gyro sensor, or the like.

The second sensor 4 is one or more sensors that detect information relating to the biological information D1b, and specifically correspond to an in-vehicle camera that captures an image including an expression and a gesture of the driver, a speech input unit that acquires speech, or the like.

The third sensor 5 is one or more sensors that detect information relating to the traffic environment information D1c, and specifically correspond to an out-of-vehicle camera that captures images of a surrounding environment including the road conditions, a distance sensor that detects the presence or absence of other vehicles in the surrounding environment and the distance to the other vehicles, a position detection sensor such as a GPS sensor, a clock circuit, a rain sensor, an in-vehicle camera or a weight sensor that detects the presence or absence of passengers, or the like.

The third sensor 5 may include a communication unit that is capable of wireless communication with the third sensor 5. The communication unit may acquire a portion of the traffic environment information D1c from an information provision server via a communication network such as the Internet. In this case, detailed road conditions, weather information and the like can be easily acquired.

The fourth sensor 6 is a driver-assistance sensor for realizing an advanced driver-assistance system (ADAS). For example, the fourth sensor 6 corresponds to a position detection sensor such as a radar sensor that detects obstacles and other vehicles, a camera that performs object recognition and lane recognition, a light detection and ranging (LiDAR) sensor, or a GPS sensor, enabling a function of automatic braking, lane maintenance support, collision alarm, and the like.

The vehicle drive unit 7 includes a drive source of the vehicle 2 and an energy supply source that supplies drive energy to the drive source. The vehicle drive unit 7 drives the drive source in accordance with a driving operation by the driver. The drive source includes, for example, at least one of a drive motor and an internal combustion engine.

The steering unit 8 includes a steering mechanism including a steering column and a steering box of the vehicle 2, and a drive source such as an electric motor for steering. The steering unit 8 activates the steering mechanism in accordance with a driving operation by the driver.

The braking unit 9 includes a brake apparatus of the vehicle 2 and a drive source such as an electric motor for braking. The braking unit 9 activates the brake apparatus in accordance with a driving operation by the driver.

The notification unit 10 includes a display and a speech output unit. Under control of the vehicle control apparatus 1, the display displays various information and the speech output unit outputs various speech inside the vehicle. This makes it possible to notify the driver and the like of various types of information. For example, the display can display a map including roads, a navigation image that guides the driver along a path from a current location to a destination, and the like. The speech output unit can output speech that guides the driver along a path.

Any conventional configuration that is capable of notifying the driver of information can be widely applied to the notification unit 10.

### 2.1 Configuration of Vehicle Control Apparatus 1

The vehicle control apparatus 1 includes a processor 21 and a memory 31.

The processor 21 functions as a computer that controls the vehicle control apparatus 1 and the vehicle 2. The processor 21 functions as an environment detection unit 22, an emotion estimation unit 23, a driving error behavior prediction unit 24, a driving influence degree prediction unit 25, and a risk suppression unit 26, by loading and executing a program 32 recorded on the memory 31.

The environment detection unit 22 includes a driving information detection unit 22a that detects the driving information D1a based on the information from the first sensor 3, a biological information detection unit 22b that detects the biological information D1b based on the information from the second sensor 4, and a traffic environment detection unit 22c that detects the traffic environment information D1c based on the information from the third sensor 5. The environment detection unit 22 is an example of a processing unit that performs the “sensing processing S1” in FIG. 1.

The emotion estimation unit 23 includes a first emotion estimation unit 23a that estimates a strong emotion based on the biological information D1b and a second emotion estimation unit 23b that estimates a strong emotion based on the traffic environment information D1c.

The emotion estimation unit 23 is an example of a processing unit that performs the “emotion estimation processing S2” in FIG. 1. In the present embodiment, the strong emotion estimated by the first emotion estimation unit 23a is denoted by reference sign D2a and the strong emotion estimated by the second emotion estimation unit 23b is denoted by reference sign D2b. The strong emotions D2a and D2b are each an example of the strong emotion D2 shown in FIG. 1.

The driving error behavior prediction unit 24 includes a first driving error behavior prediction unit 24a that predicts driving error behavior D3 by using a statistical database 34 stored in the memory 31 when the strong emotion D2 has been estimated, and a second driving error behavior detection unit 24b that detects the driving error behavior D3c by using the driving information D1a.

The driving error behavior prediction unit 24 is an example of a processing unit that performs the “driving error behavior prediction processing S3” in FIG. 1. In the present embodiment, it is determined whether the driving error behavior D3a and D3b in FIG. 1 is included, based on the driving error behavior D3 predicted by the first driving error behavior prediction unit 24a. However, it may also be determined whether the driving error behavior D3a and D3b is included, based on the driving error behavior D3c detected by the second driving error behavior detection unit 24b.

The driving influence degree prediction unit 25 performs processing of predicting a degree of influence on future driving, based on the predicted driving error behavior D3a and D3b. The driving influence degree prediction unit 25 includes a driving risk prediction unit 25a that predicts the driving risk (the occurrence probability D4) as one degree of influence on the future driving. The driving influence degree prediction unit 25 is an example of a processing unit that performs the “driving risk prediction processing S4” in FIG. 1.

The risk suppression unit 26 includes a control content determination unit 26a that determines control content of the vehicle 2 based on a prediction result of the driving influence degree prediction unit 25, a vehicle control unit 26b that performs control corresponding to the determined control content, and a notification control unit 26c that performs notification corresponding to the determined control content.

The risk suppression unit 26 is an example of a processing unit that performs the “driving risk suppression processing S5” in FIG. 1.

Other than the program 32, the memory 31 stores map data 33, the statistical database 34, and a position database 35. The map data 33 is map data for navigation and includes, for example, facility data consisting of nodes and links indicating road shapes, and point of interest (POI) data in which names, positions, and the like of facilities that may be destination candidates are stated. The map data 33 may be information that is acquired as appropriate via a communication network.

The statistical database 34 includes multiple types of statistical data used by the emotion estimation unit 23, the driving error behavior prediction unit 24, and the driving influence degree prediction unit 25. To be specific, the statistical database 34 includes an environment-emotion table T1, and a biology-emotion table consisting of an expression-emotion table T2a and a gesture-emotion table T2b, driving error behavior statistical data T3 indicating statistics of driving error behavior involving human error when experiencing a specific emotion, driving risk statistical data T4 indicating the occurrence probability D4 of the driving error behavior causing the crash event and the near-crash event, and the like, each to be described below.

The position database 35 stores a travel position and the like at the time of the emotion estimation unit 23 estimating a specific strong emotion D2. The travel position is position information detected by the position detection sensor such as the GPS sensor included in the third sensor 5. The vehicle control apparatus 1 (for example, the emotion estimation unit 23) records this travel position.

### 3. Operation of Vehicle Control Apparatus 1

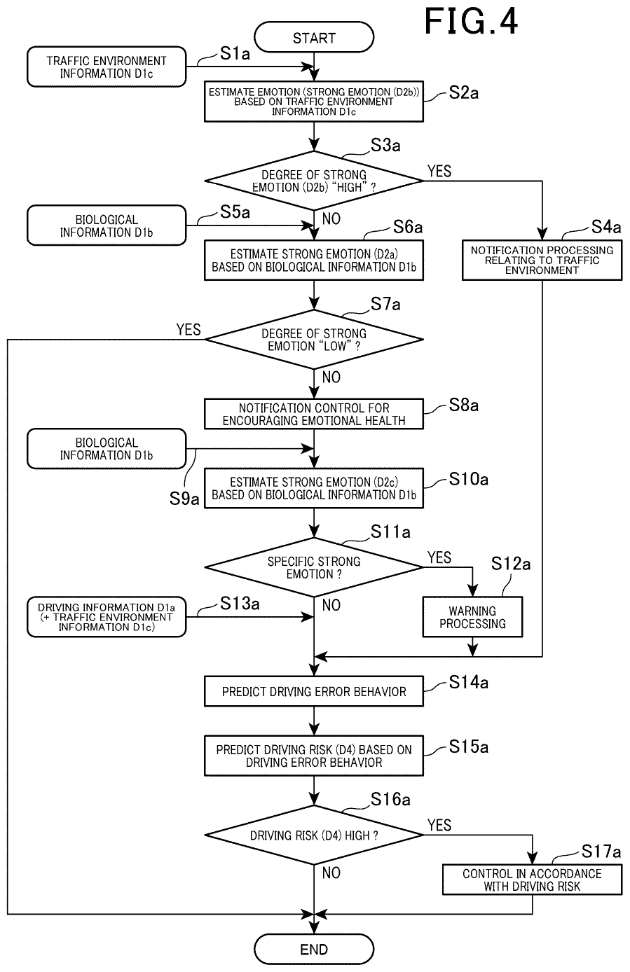

An operation of the vehicle control apparatus 1 will be described in accordance with a flowchart shown in FIG. 4.

The vehicle control apparatus 1 repeatedly executes processing shown in FIG. 4 while the vehicle 2 is in traveling.

First, the vehicle control apparatus 1 acquires the traffic environment information D1c via the traffic environment detection unit 22c (step S1a), and estimates an emotion including a strong emotion D2b based on the acquired traffic environment information D1c via the second emotion estimation unit 23b (step S2a).

In the present embodiment, “static traffic environment information” and “dynamic traffic environment information” are acquired as the traffic environment information D1c in step S1a.

The “static traffic environment information” is information that allows identification of a region where the vehicle 2 is located, road conditions, how the driver grips the steering wheel, time information indicating daytime or nighttime, in-vehicle environment information indicating the presence or absence of passengers, weather information indicating the weather and the like. The “dynamic traffic environment information” is described below.

### 3.1 Estimation of Strong Emotion Based on Static Traffic Environment Information

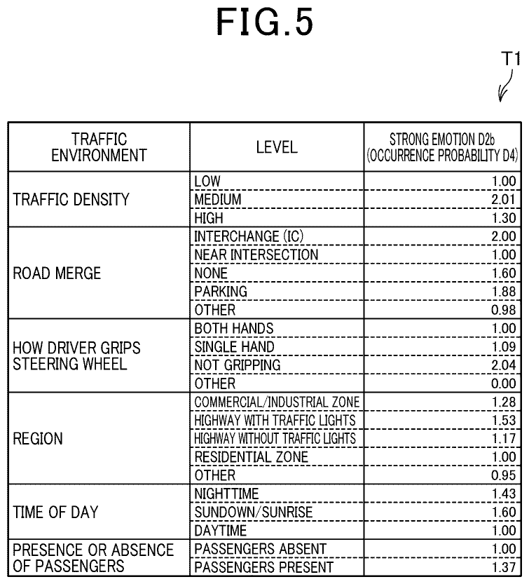

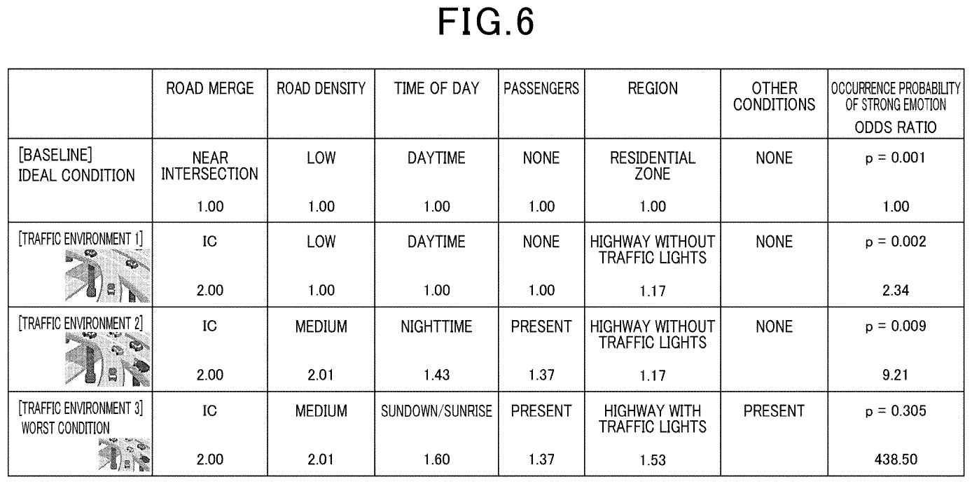

An estimation method of the strong emotion D2b based on the “static traffic environment information” will be described with reference to FIG. 5 and FIG. 6.

FIG. 5 shows odds ratios indicating a degree of the strong emotion D2b for each condition included in the “static traffic environment information”. These odds ratios are each an example of an index indicating how much higher a probability is of the driver experiencing a strong emotion as compared to a baseline (ideal condition). The degree of the strong emotion D2b can also be interpreted as an index of the occurrence probability D4.

As shown in FIG. 6, it is possible to calculate odds ratios indicating the strong emotion D2b corresponding to the static traffic environment information, by calculating a combined value of odds ratios for multiple conditions included in the static traffic environment information. Reference sign p in FIG. 6 indicates an occurrence probability of the strong emotion at baseline.

In FIG. 6, the degree of the strong emotion D2b increases in the order of baseline, a traffic environment 1, a traffic environment 2, and a traffic environment 3, and the probability that the driver is experiencing a strong emotion increases.

In the present embodiment, the second emotion estimation unit 23b acquires the odds ratios of the strong emotion D2b for each environment based on the environment-emotion table T1 in which the traffic environment and the odds ratios of the strong emotion D2b shown in FIG. 5 are associated with each other, and calculates the strong emotion D2b corresponding to the static traffic environment information acquired in step S1a by using a calculation formula for combining each odds ratio.

### 3.2 Dynamic Traffic Environment Information

The “dynamic traffic environment information” is categorized into “integral triggers” being triggers related to driving and “incidental triggers” being triggers not related to driving. The “integral triggers” are information relating mainly to behavior other than behavior outside of one's own vehicle. To be specific, the information corresponds to low-speed driving of a vehicle in front, cutting off, reckless driving of a rear vehicle, or a situation in which the brake of one's own vehicle must be applied when an oncoming vehicle is about to turn left in front of one's own vehicle.

Note that as the estimation method of the strong emotion D2b based on the information relating to this behavior other than the behavior outside of one's own vehicle, the odds ratios of the strong emotion D2b may be calculated using statistical values such as average values or maximum values of a speed, acceleration, relative speed to a vehicle in front, and an inter-vehicular time of one's own vehicle.

The “incidental triggers” are information relating mainly to behavior other than behavior of the driver inside the vehicle. To be specific, the information corresponds to conversations with passengers, telephone use, and in-vehicle music.

In the present embodiment, the second emotion estimation unit 23b multiplies the occurrence probability D4 of the strong emotion D2b calculated using the static traffic environment information by a predetermined coefficient (a value of one or higher) set based on at least one of the “integral triggers” and the “incidental triggers”, or calculates the strong emotion D2b taking into account the dynamic traffic environment information by adding up the odds ratios of the strong emotion D2b calculated using the dynamic traffic environment information. This improves estimation accuracy of the strong emotion as compared to when not taking into account the dynamic traffic environment information.

Note that the coefficient for the “integral triggers” is set larger than the coefficient for the “incidental triggers”, so that the “integral triggers” are more likely to be determined as strong emotions.

However, the present invention is not limited to estimating the strong emotion by using both the “static traffic environment information” and the “dynamic traffic environment information”, but may also estimate the strong emotion by using only the static traffic environment information.

### 3.3 Notification and Control Based on Strong Emotion

Returning to FIG. 4, the vehicle control apparatus 1 determines the degree of the strong emotion D2b estimated based on the traffic environment information D1c via the second emotion estimation unit 23b (step S3a).

In the present embodiment, it is determined whether the degree of the strong emotion D2b is “high”. In this case, the degree of the strong emotion D2b may be determined in multiple levels such as high, medium and low, it may be determined whether the degree of the strong emotion D2b corresponds to “high”, and it may be determined that the degree of the strong emotion D2b is “high” when the degree of strong emotion D2b is equal to or higher than a first predetermined value. When the degree of the strong emotion D2b is “high” (step S3a: YES), the vehicle control apparatus 1 performs notification processing relating to the traffic environment via the notification control unit 26c (step S4a). The first predetermined value may be set to an appropriate value at which the degree of the strong emotion D2b can be determined to be “high”, in other words, an appropriate value at which the notification processing relating to the traffic environment can be appropriately executed.

In the notification processing of step S4a, notification is performed in order to call attention to the traffic environment. For example, the driver is notified through the notification unit 10 of content such as “Beware of heavy traffic and the possibility of being cut off from the side”.

This processing is an example of processing of “performing predetermined notification without using the prediction result of the driving error behavior prediction unit” according to the present disclosure. After this notification processing ends, the flow transitions to the processing of step S14a.

On the other hand, when the degree of the strong emotion D2b estimated in step S3a is not “high” (step S3a: NO), the vehicle control apparatus 1 acquires the biological information D1b via the biological information detection unit 22b (step S5a) and estimates the strong emotion D2a based on the biological information D1b via the first emotion estimation unit 23a (step S6a).

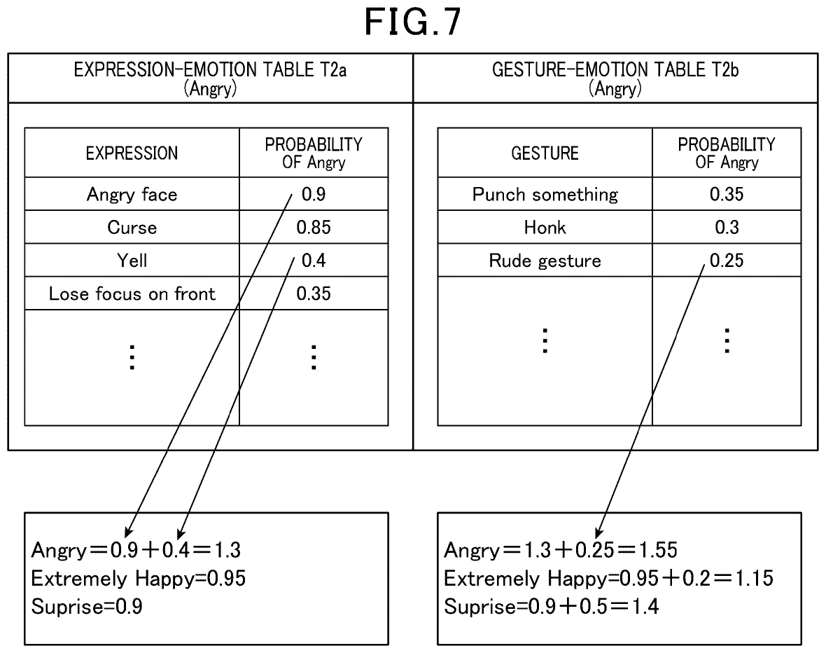

In step S6a, it is determined if an expression of the driver included in the biological information D1b corresponds to a strong emotion, by analyzing the expression. The expression-emotion table T2a in the statistical database 34 stored in the memory 31 is referenced for this determination. The expression-emotion table T2a includes data in which an expression and the occurrence probability of a strong emotion (for example, “angry”) are associated with each other, and is shown in FIG. 7.

For example, when “angry face” and “Yell” are detected as expressions, a value obtained by combining each probability is calculated (for example, 0.9+0.4=1.3).

Similarly, a value obtained by combining each probability is calculated, by using the “expression-emotion table T2a” corresponding to another strong emotion (for example, “extremely happy” or “surprise”). For example, it is determined that “angry” having the highest value is dominant when calculating that the combined value in the case of “angry” is 1.3, the combined value in the case of “extremely happy” is 0.95, and the combined value in the case of “Surprise” is 0.9. These combined values each correspond to the “degree of emotion (emotional level)”.

In step S6a, a gesture of the driver included in the biological information D1b is similarly analyzed. The “gesture-emotion table T2b” is referenced when determining if the gesture corresponds to a strong emotion. The “gesture-emotion table T2b” includes data in which a gesture and the occurrence probability of a strong emotion are associated with each other, and is shown in FIG. 7.

For example, when “rude gesture” is detected, it is determined from the “gesture-emotion table T2b” shown in FIG. 7 that the probability of “angry” is 0.25.

A probability value acquired based on the gesture is combined with a probability value based on the expression. For example, when the probability of “angry” is calculated to be 1.3 based on the expression, a final value is 1.55 by adding 0.25 based on the gesture, as shown in FIG. 7. Similarly, a final value for “extremely happy” is 1.15 by adding 0.2 to 0.95, and a final value for “surprise” is 1.4 by adding 0.5 to 0.9. As a result, the degree of “angry” is determined to be the highest.

The most dominant strong emotion is determined taking into account facial expressions and gestures, based on corrected probability values. In the present embodiment, accuracy can be improved by applying correction coefficients as necessary.

It is also possible to categorize strong emotions based on the expression of the driver, and to evaluate the degree of the strong emotions based on the gestures of the driver. For example, when the probability of “angry” is identified to be 1.3 based on the expression, a correction value (for example, 0.25) is taken into account due to the gesture and the emotion level is determined to be “medium”. A method can be considered of setting, as this determination criterion, “low” if the probability of the gesture is less than 0.1, “medium” if the probability is between 0.1 and 0.3, and “high” if the probability is equal to or higher than 0.3, but this is a freely-selected criterion.

Returning to FIG. 4, the vehicle control apparatus 1 determines whether the degree of the strong emotion D2a estimated in step S6a is “low” (step S7a). When it is determined that the degree of the strong emotion D2a is “high” or “medium” as a result of the determination (step S7a: NO), the notification control unit 26c performs notification control for encouraging emotional health (step S8a). On the other hand, when the degree of the strong emotion D2a is “low” (step S7a: YES), the processing of the flowchart shown in FIG. 4 ends.

Note that step S7a is not limited to processing of determining whether the degree of the strong emotion D2a is “low” from among multiple levels such as high, medium, and low, but it may also be determined that the degree of strong emotion D2b is “low” when the degree of strong emotion D2b is equal to or lower than a second predetermined value, or it may be determined whether the degree of the strong emotion D2a is “low” by taking into account the odds ratio of strong emotion D2b. For example, the determination in step S7a may be performed based on a resulting value of the degree of the strong emotion D2a+β (correction coefficient)×the strong emotion D2b. The second predetermined value may be set to an appropriate value at which the degree of the strong emotion D2b can be determined to be “low”, in other words, an appropriate value at which it is determined whether to perform the notification control of step S8a.

In step S8a, the notification control is performed for decreasing the degree of the strong emotion or to guide the driver to a normal emotional state. To be specific, the notification control corresponds to control for outputting a sound or image to allow the driver to relax, a sound or image to calm down the driver, or a sound or image to distract the driver. These sounds include dialogue, music, and the like.

Subsequently, the vehicle control apparatus 1 acquires the biological information D1b again via the biological information detection unit 22b (step S9a). Next, the vehicle control apparatus 1 estimates the strong emotion D2a based on the acquired biological information D1b via the first emotion estimation unit 23a (step S10a). The processing of step S10a is similar to that of step S6a, and the most dominant strong emotion at this point is identified.

Next, the vehicle control apparatus 1 determines whether the strong emotion estimated via the first emotion estimation unit 23a is a specific strong emotion for which the driving risk is relatively high (step S11a). The specific strong emotion for which the driving risk is high is “angry”, “extremely happy”, or the like, as shown in FIG. 2.

When it is determined that the estimated strong emotion is the specific strong emotion for which the driving risk is high (step S11a: YES), the vehicle control apparatus 1 performs alarm processing via the notification control unit 26c (step S12a). In the alarm processing, processing is performed for decreasing the degree of the strong emotion or to guide the driver to a normal emotional state.

In the present embodiment, the alarm processing is performed in accordance with the category and degree of the strong emotion. To be specific, when the specific strong emotion is “angry” and the degree of the strong emotion is “high”, a sound or image is output at a lower volume or using language that the driver can easily relate to. When the specific strong emotion is “angry”, a conspicuous sound or image is output when the degree of the strong emotion is “medium”, and a subdued sound or image is output when the degree of the strong emotion is “low”.

After performing the alarm processing of step S12a or when it is determined in step S11a that the estimated strong emotion is not the specific strong emotion (step S11a: NO), the vehicle control apparatus 1 acquires the driving information D1a and the traffic environment information D1c (step S13a), and predicts future driving error behavior via the second driving error behavior detection unit 24b and the first driving error behavior prediction unit 24a (step S14a).

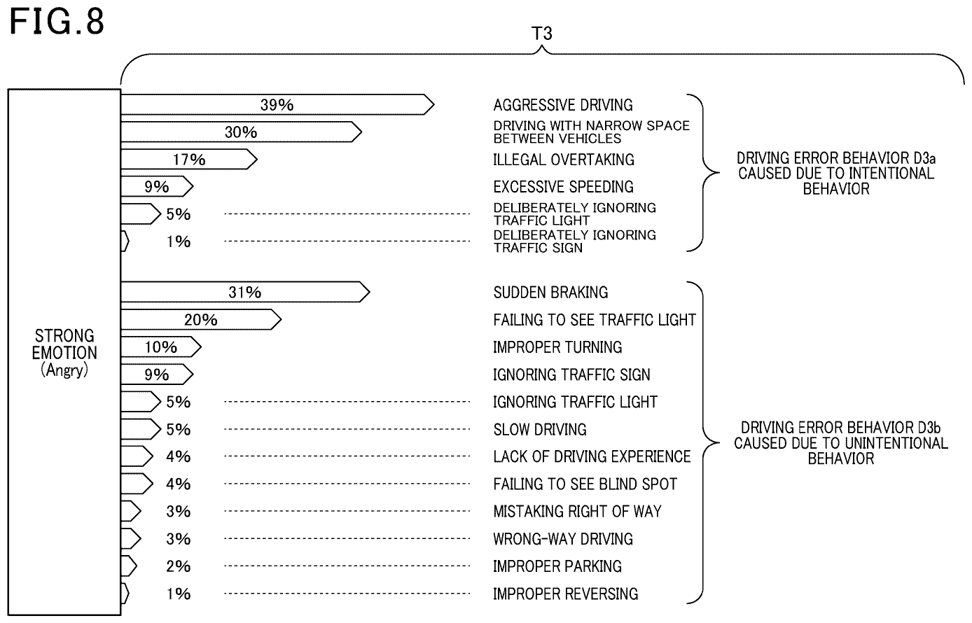

The detection of the driving error behavior by the second driving error behavior detection unit 24b is processing of identifying real-time or past driving error behavior D3c using the driving information D1a. Since the driving information D1a includes information indicating the real-time or past driving conditions of the driver, the second driving error behavior detection unit 24b detects the real-time or past driving error behavior D3c of the driver using the real-time or past driving conditions, and identifies the identified driving error behavior D3c as future driving error behavior. To be specific, the second driving error behavior detection unit 24b detects the driving error behavior D3a and D3b (see FIG. 8) involving human error when experiencing the strong emotion.

A prediction method for the driving error behavior D3 by the first driving error behavior prediction unit 24a will be described with reference to FIG. 8.

FIG. 8 shows the probability that driving error behavior involving human error occurs when experiencing the strong emotion “angry”. Driving error behavior involving human error can be categorized into the driving error behavior D3a caused due to intentional behavior of the driver and the driving error behavior D3b caused due to unintentional behavior of the driver.

The driving error behavior D3a caused due to intentional behavior of the driver specifically corresponds to aggressive driving, driving with a narrow space between vehicles, and the like. The driving error behavior D3b caused due to unintentional behavior of the driver specifically corresponds to sudden braking, overlooking traffic lights, and the like.

In the present embodiment, data that allows probabilistic identification of the driving error behavior D3a and D3b involving human error for each strong emotion is included in the statistical database 34 as the “driving error behavior statistical data T3”. The “driving error behavior statistical data T3” is data created based on the statistical data shown in FIG. 8 and the like. For example, the driving error behavior statistical data T3 is data in which the driving error behavior D3a and D3b involving human error and the occurrence probabilities of the driving error behavior D3a and D3b are respectively associated with each other for each strong emotion.

It is possible to predict, as future driving error behavior, the driving error behavior D3a and D3b for which the occurrence probabilities are relatively high based on the strong emotion, by using the “driving error behavior statistical data T3”.

Furthermore, it is possible to easily identify if future driving error behavior is the driving error behavior D3a caused due to intentional behavior of the driver or the driving error behavior D3b caused due to unintentional behavior of the driver, by making the “driving error behavior statistical data T3” into data that allows identification of whether the future driving error behavior is the driving error behavior D3a caused due to intentional behavior of the driver or the driving error behavior D3b caused due to unintentional behavior of the driver.

Returning to FIG. 4, the vehicle control apparatus 1 predicts the driving risk (the occurrence probability D4) based on the predicted driving error behavior via the driving risk prediction unit 25a (step S15a).

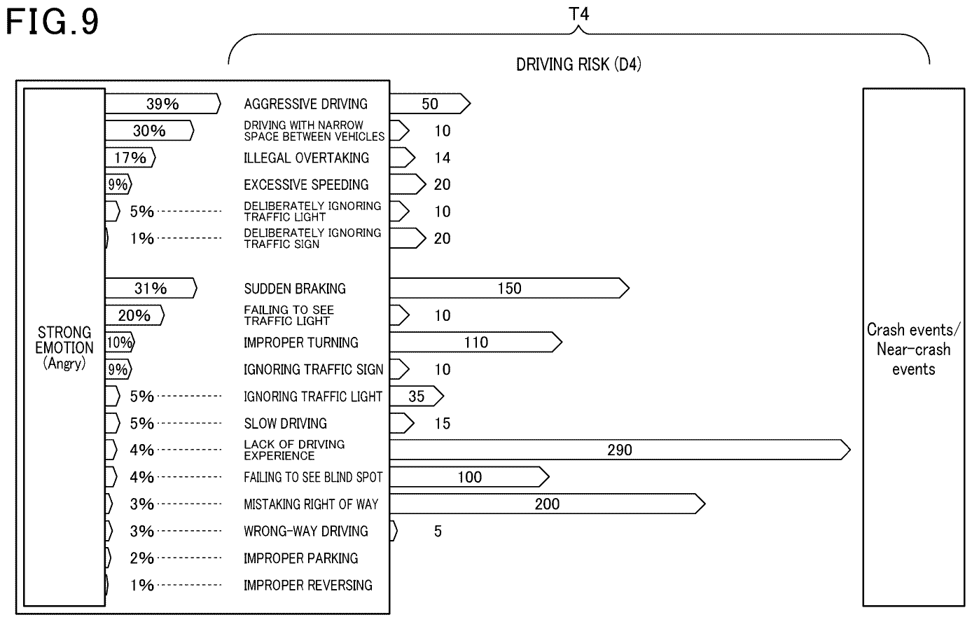

An example of a prediction method for the driving risk will be described with reference to FIG. 9.

FIG. 9 shows the driving risk (the occurrence probability D4) of the driving error behavior causing the situation to be avoided (crash event and near-crash event) when experiencing the strong emotion “angry”. FIG. 9 shows the occurrence probability D4 as an odds ratio.

In the present embodiment, data in which the driving error behavior D3a and D3b involving human error and the driving risk (the occurrence probability D4) are associated with each other for each strong emotion is included in the statistical database 34 as the “driving risk statistical data T4”.

The “driving risk statistical data T4” is data created based on the statistical data shown in FIG. 9 and the like. For example, the driving risk statistical data T4 is data in which the driving error behavior D3a and D3b involving human error and the driving risk (the occurrence probability D4) are associated with each other for each strong emotion.

It is possible to easily identify the driving risk (the occurrence probability D4) respectively associated to the predicted driving error behavior D3a and D3b based on the strong emotion, by using the “driving risk statistical data T4”.

Returning to FIG. 4, the vehicle control apparatus 1 determines whether the predicted driving risk is higher than a predetermined threshold value via the driving risk prediction unit 25a (step S16a). When the driving risk is higher (step S16a: YES), control is performed in accordance with the driving risk by the risk suppression unit 26 (step S17a).

To be specific, in step S17a, the control content determination unit 26a identifies if the driving error behavior for which the driving risk is high is the driving error behavior D3a caused due to intentional behavior of the driver or the driving error behavior D3b caused due to unintentional behavior of the driver, and determines control content in accordance with the identified driving error behavior. In the case of the driving error behavior D3a, warning processing is performed of suppressing judgment errors by notifying the driver of predicted future trouble and the damage caused thereby, via the notification control unit 26c. For example, in the case of aggressive driving, a notification such as “Reckless driving may result in criminal penalties. Please refrain from reckless driving.” may be provided.

In the case of the driving error behavior D3b, ADAS control for avoiding predicted future trouble is performed via the vehicle control unit 26b. To be specific, control of the vehicle 2 is performed for adjusting vehicle speed to an appropriate range, for maintaining the distance between vehicles through brake control, for preventing lane departure through lane assistance support, and the like.

Note that data structures and the like of the environment-emotion table T1, the driving risk statistical data T4, the expression-emotion table T2a, the gesture-emotion table T2b, the driving error behavior statistical data T3, and the driving risk statistical data T4 may be changed as appropriate.

The above is the operation of the vehicle control apparatus 1 based on the flowchart shown in FIG. 3.

Note that the processing of steps S4a, S8a, S12a, and S17a, which is processing performed by the risk suppression unit 26, is an example of the “predetermined control” according to the present disclosure. The content of this processing may be changed as appropriate.

For example, the risk suppression unit 26 may perform, via the vehicle control unit 26b, route guidance processing of guiding the driver along an appropriate route by using navigation technology, or state restoration processing of restoring the state of the driver. When performing the route guidance processing, in step S4a, performing route guidance processing so as to select a traffic environment for reducing the degree of the strong emotion D2b in step S4a, performing route guidance processing suitable for restoring the emotional state in step S8a, and performing route guidance processing suitable for suppressing driving error behavior with a high driving risk in step S17a can be considered.

The state restoration processing is processing of maintaining or improving the driver's concentration through verbal communication, smell, music, and the like.

In the case of a specific strong emotion for which the driving risk is relatively high, for example, a specific strong emotion for which the driving error behavior is predicted to have a relatively high driving risk, a position of the vehicle 2 is stored in the position database 35, and the risk suppression unit 26 performs route guidance processing of guiding the driver along a route that avoids the stored position when the driving risk prediction unit 25a predicts that the driver will pass the position stored in the position database 35 based on information about a travel route that the driver has set.

In the present embodiment, the processing of the environment detection unit 22 is an example of an environment detection step according to the present disclosure. The processing of the emotion estimation unit 23 is an example of an emotion estimation step according to the present disclosure. The processing of the driving error behavior prediction unit 24 is an example of a driving error behavior prediction step according to the present disclosure. The processing of the driving risk prediction unit 25a is an example of a driving risk prediction step according to the present disclosure. The processing of the risk suppression unit 26 is an example of a risk suppression step according to the present disclosure. The processing of the control content determination unit 26a is an example of a control content determination step according to the present disclosure. The processing of the vehicle control unit 26b is an example of a vehicle control step according to the present disclosure. The processing of the notification control unit 26c is an example of a notification control step according to the present disclosure.

### 4. Operation and Effects

### 4.1 Overall Algorithm

As described above, the vehicle control apparatus 1 according to the present embodiment includes the environment detection unit 22 that detects the environment information D1 about the interior and the exterior of the target vehicle, the emotion estimation unit 23 that estimates the emotion D2 of the driver based on the environment information D1, the driving error behavior prediction unit 24 that predicts the driving error behavior D3a and D3b of the driver when the estimated emotion D2 is a strong emotion, the driving risk prediction unit 25a that predicts the driving risk based on the predicted driving error behavior D3a and D3b, and the risk suppression unit 26 that performs the predetermined control in accordance with the predicted driving risk.

According to this configuration, high-accuracy prediction of the driving risk is possible by predicting the driving error behavior, when the driver is experiencing a strong emotion. The high-accurate prediction enables appropriate control in accordance with the driving risk. This makes it possible to effectively prevent an unpredicted situation during driving by improving the prediction accuracy of the driving risk.

Note that a case has been described in which the environment information D1 about the interior and the exterior of the target vehicle is detected and the emotion D2 of the driver based on the detected environment information D1 is detected, but the environment information D1 about at least one of the interior and the exterior of the target vehicle may be detected and the emotion D2 of the driver may be estimated based on the detected environment information D1.

The environment detection unit 22 includes the biological information detection unit 22b that detects the biological information D1b of the driver as the environment information D1, and the traffic environment detection unit 22c that detects the traffic environment information D1c of the target vehicle as the environment information D1. The emotion estimation unit 23 estimates the strong emotions based on each of the biological information D1b of the driver and the traffic environment information D1c.

According to this configuration, it is possible to improve the estimation accuracy of the strong emotion, which improves the prediction accuracy of the driving risk and facilitates more appropriate control and the comfortability of suppressing excessive control.

The environment information D1 includes the driving information D1a of the driver in the target vehicle. The driving error behavior prediction unit 24 predicts the driving error behavior of the driver based on the real-time driving conditions including the driving error behavior statistical data T3 when experiencing a strong emotion and the driving error behavior D3c detected using the driving information D1a.

According to this configuration, it is possible to improve the prediction accuracy of the driving error behavior, which improves the prediction accuracy of the driving risk and facilitates more appropriate control and the comfortability of suppressing excessive control.

The driving risk prediction unit 25a predicts the driving risk based on the driving risk statistical data T4 that allows identification of the relationship between the driving error behavior and the situation to be avoided (crash event/near-crash event). According to this configuration, it is possible to improve the prediction accuracy of the driving risk causing the situation to be avoided. Note that the driving risk statistical data T4 is an example of statistical data that allows identification of the relationship between the driving error behavior and the situation to be avoided. The situation to be avoided may be changed as appropriate.

The risk suppression unit 26 performs, as the predetermined control, at least one of the vehicle speed control, the brake control, the route guidance processing, the state restoration processing, and the warning processing in accordance with the driving risk. It is possible to directly reduce the driving risk directly by performing the vehicle speed control and the brake control, and to indirectly reduce the driving risk through the route guidance processing, the state restoration processing, and the warning processing. This makes it possible to appropriately control the target vehicle in accordance with the predicted driving risk, thereby reducing the driving risk.

In the present embodiment, it is possible to effectively reduce the driving risk by performing, as the predetermined control, control of suppressing driving error behavior with a high driving risk among the driving error behavior predicted by the driving error behavior prediction unit 24.

The vehicle control apparatus 1 includes the memory 31 that stores the position of the target vehicle when the estimated emotion is a specific strong emotion. When it is predicted via the driving risk prediction unit 25a that the target vehicle will pass through the position stored in the memory 31, the risk suppression unit 26 performs the route guidance processing of guiding the driver along a route for avoiding the stored position. According to this configuration, it is possible to guide the driver along a route for avoiding a position where the driver has experienced a strong emotion in the past, making it possible to avoid a situation in which the driving risk increases. Note that the memory 31 corresponds to a storage unit that stores the position of the target vehicle, and any recording medium can be applied thereto.

Among the driving error behavior D3a and D3b predicted by the driving error behavior prediction unit 24, the risk suppression unit 26 performs the notification processing of notifying the driver as the predetermined control in the case of the driving error behavior D3a caused due to intentional behavior of the driver, whereas the risk suppression unit 26 performs the driving control of the target vehicle as the predetermined control in the case of the driving error behavior D3b caused due to unintentional behavior of the driver.

According to this configuration, it is possible to change the control in accordance with whether the predicted driving error behavior is the driving error behavior performed intentionally by the driver, enabling appropriate control in accordance with the driving error behavior.

### 4.2 Emotion Estimation

In the present embodiment, the vehicle control apparatus 1 includes the biological information detection unit 22b that detects the biological information D1b of the driver of the target vehicle, the environment detection unit 22 that detects the environment information D1 in a surrounding area of the target vehicle, and the emotion estimation unit 23 that estimates the strong emotion of the driver based on the biological information D1b and the environment information D1. According to this configuration, it is possible to provide an emotion estimation apparatus that facilitates quick and appropriate estimation of the emotions influencing the driving error behavior of the driver. The emotion estimation apparatus may have only the functions of the vehicle control apparatus 1 related to emotion estimation, or may combine these functions with functions not related to the emotion estimation.

The emotion estimation unit 23 estimates the strong emotion using the environment information D1, based on the environment-emotion table T1 indicating the relationship between the environment surrounding the vehicle and the strong emotion. According to this configuration, it is possible to efficiently identify the strong emotion from the environment surrounding the vehicle.

The environment information D1 includes at least one of the road conditions, traffic density, the time of day, the region, and the presence or absence of passengers. According to this configuration, it is possible to estimate the strong emotions influenced by at least one of the road conditions, the traffic density, the time of day, the region, and the presence or absence of passengers.

The emotion estimation unit 23 estimates the strong emotion using the biological information D1b of the driver, based on the biology-emotion table (the expression-emotion table T2a and the gesture-emotion table T2b) indicating the relationship between the biological information of the driver and the strong emotion. According to this configuration, it is possible to efficiently identify the strong emotion using the biological information D1b.

The biological information D1b includes information that allows identification of each of the expressions and gestures of the driver. According to this configuration, it is possible to efficiently identify the strong emotion from the emotions and the gestures.

The emotion estimation unit 23 estimates the strong emotion, by using the driving risk statistical data T4 being statistical data that allows identification of the relationship between the surrounding environment of the target vehicle and the strong emotion, and the driving error behavior detected from the driving information D1a of the driver in the target vehicle. According to this configuration, the estimation accuracy for the strong emotion is improved.

### 4.3 Driving Prediction

In the present embodiment, the vehicle control apparatus 1 includes the biological information detection unit 22b that detects the biological information D1b of the driver of the target vehicle, the traffic environment detection unit 22c that detects the traffic environment information D1c being environment information about the surrounding area of the target vehicle, the emotion estimation unit 23 that estimates the strong emotion of the driver based on the biological information D1b of the driver and the traffic environment information D1c, the second driving error behavior detection unit 24b that detects the driving error behavior D3c of the driver, and the driving influence degree prediction unit 25 that predicts the degree of influence on future driving error behavior based on the estimation result of the strong emotion of the driver and the detection result of the driving error behavior D3c.

According to this configuration, quick and appropriate estimation of the strong emotion influencing the driving error behavior of the driver by using the biological information D1b and the traffic environment information D1c is facilitated. By taking into account the relationship between the estimation result of the strong emotion and the driving error behavior of the driver, it is possible to provide a driving prediction apparatus that can predict the degree of influence on future driving error behavior with high accuracy. Note that the driving prediction apparatus can also be referred to as a driving influence degree prediction apparatus.

Note that in the present embodiment, a case has been exemplified in which the driving influence degree prediction unit 25 predicts driving risk as the degree of influence on future driving error behavior, but the present invention does not have to be limited to the driving risk.

In the present embodiment, the vehicle control apparatus 1 includes the notification control unit 26c that performs the predetermined notification based on the prediction result of the driving influence degree prediction unit 25. According to this configuration, it is possible to perform appropriate notification in accordance with the degree of influence on future driving error behavior and to effectively prevent an unpredictable situation during driving.

The second driving error behavior detection unit 24b detects the driving error behavior D3c using the driving information D1a of the driver. According to this configuration, it is possible to detect the driving error behavior D3c of the driver with high accuracy, and as a result, it is possible to predict the degree of influence on future driving error behavior with high accuracy.

### 4.4 Vehicle Control Based on Emotion

In the present embodiment, the vehicle control apparatus 1 includes the environment detection unit 22 that detects the environment information D1 about at least one of the interior and the exterior of the target vehicle, the emotion estimation unit 23 that estimates the emotion D2 of the driver of the target vehicle based on the environment information D1, the control content determination unit 26a that determines the control content of the target vehicle when the estimated emotion D2 is a strong emotion, and the vehicle control unit 26b that performs control of the target vehicle based on the determined control content.

According to this configuration, it is possible to provide the vehicle control apparatus 1 for controlling the vehicle in accordance with a strong emotion influencing future driving error behavior, by determining the control content of the target vehicle, when the driver is experiencing a strong emotion. This makes it possible to take appropriate measures in accordance with future driving error behavior of the driver.

In the present embodiment, a case has been exemplified in which the driving error behavior is predicted and control is performed of the target vehicle in accordance with the driving risk based on the predicted driving error behavior when the estimated emotion D2 is a strong emotion, but the prediction of driving error behavior, the prediction of the driving risk, or the like may be omitted. In other words, when the estimated emotion D2 is a strong emotion, determining the control content of the target vehicle can be widely applied as appropriate to the extent that the vehicle can be controlled in accordance with the strong emotion influencing the future driving error behavior.

The control content determination unit 26a determines the control content in accordance with at least one of the degree of the strong emotion and the category of the strong emotion. According to this configuration, it is possible to appropriately change the content of the control performed when experiencing a strong emotion.

The environment information D1 includes the traffic environment information D1c of the target vehicle and the biological information D1b of the driver. The emotion estimation unit 23 estimates the emotion D2 based on each of the traffic environment information D1c and the biological information D1b. The control content determination unit 26a performs processing of determining the control content in accordance with the degree of the strong emotion estimated based on the traffic environment information D1c and processing of determining the control content in accordance with the category of the strong emotion estimated based on the biological information D1b. According to this configuration, it is possible to change the control content, a priority of the control, and the like performed when experiencing a strong emotion based on the traffic environment, the biological information D1b, and the like, making it possible to take appropriate measures.

The vehicle control apparatus 1 includes the driving error behavior prediction unit 24 that predicts the driving error behavior D3a and D3b of the driver, when the estimated emotion D2 is a strong emotion. Among the driving error behavior D3a and D3b predicted by the driving error behavior prediction unit 24, the control content determination unit 26a sets the control content of the target vehicle as the notification processing of notifying the driver in the case of the driving error behavior D3a caused due to intentional behavior of the driver, whereas the control content determination unit 26a sets the control content of the target vehicle as the driving control of the target vehicle in the case of the driving error behavior D3b caused due to unintentional behavior of the driver. According to this configuration, it is possible to appropriately change the control content in accordance with whether the predicted driving error behavior is the driving error behavior performed intentionally by the driver.

### 4.5 Vehicle Control When Environment Increases Degree of Strong Emotion

In the present embodiment, the vehicle control apparatus 1 includes the environment detection unit 22 that detects the environment information D1 about at least one of the interior and the exterior of the target vehicle, the emotion estimation unit 23 that estimates the strong emotion of the driver based on the environment information D1, the driving error behavior prediction unit 24 that predicts the driving error behavior of the driver, and the risk suppression unit 26 that performs the predetermined control for avoiding a situation to be avoided based on the prediction result of the driving error behavior prediction unit 24. The risk suppression unit 26 performs the predetermined control without using the prediction result of the driving error behavior prediction unit 24, when the degree of the strong emotion estimated by the emotion estimation unit 23 is high (steps S1a to S4a).

According to this configuration, it is possible perform appropriate control by using the prediction result about the driving error behavior of the driver. When the environment is such that the degree of the strong emotion of the driver increases, it is possible to quickly perform the predetermined control, since the predetermined control is performed without using the prediction result about driving error behavior. This makes it possible to quickly take measures in accordance with the situation.

The emotion estimation unit 23 includes the first emotion estimation unit 23a that estimates the strong emotion D2a based on the biological information D1b of the driver constituting a portion of the environment information D1 and the second emotion estimation unit 23b that estimates the strong emotion D2b based on the traffic environment information D1c constituting a portion of the environment information D1. The risk suppression unit 26 performs the predetermined control without the first emotion estimation unit 23a estimating the strong emotion D2a, when the degree of the strong emotion D2b estimated by the second emotion estimation unit 23b is high.

According to this configuration, since it is possible to estimate each of the strong emotion D2a based on the biological information D1b and the strong emotion D2b based on the traffic environment information D1c, it is possible to perform the predetermined control in accordance with each estimation result. When the degree of the strong emotion estimated based on the traffic environment information D1c is high, it is possible to quickly perform the predetermined control, since the predetermined control is performed without estimating the strong emotion D2a based on the biological information D1b of the driver. Therefore, when the traffic environment is such that the degree of the strong emotion increases, it is possible to quickly take measures.

When the degree of the strong emotion D2b estimated by the second emotion estimation unit 23b is high, the risk suppression unit 26 performs processing of calling attention to the traffic environment as the predetermined control. According to this configuration, it is possible to quickly perform appropriate notification.

When the degree of the strong emotion D2b estimated by the second emotion estimation unit 23b is not higher than the first predetermined value, the first emotion estimation unit 23a estimates the strong emotion D2a of the driver based on the biological information D1b. When the degree of the estimated strong emotion D2a is not lower than the second predetermined value, the risk suppression unit 26 performs, as the predetermined control, the notification control for reducing the degree of the strong emotion.

According to this configuration, when the degree of the strong emotion D2a estimated based on the biological information D1b is not lower than the second predetermined value, it is possible to encourage emotional health by performing the notification control for reducing the degree of the strong emotion.

After performing the notification control for reducing the degree of the strong emotion, the first emotion estimation unit 23a estimates the strong emotion D2a of the driver based on the biological information D1b, and when the estimated strong emotion D2a is a specific strong emotion, the risk suppression unit 26 performs the alarm processing in accordance with the category and the degree of this strong emotion (step S12a).

According to this configuration, when the strong emotion estimated based on the biological information D1b is a specific strong emotion after encouraged emotional health, it is possible to simplify or eliminate the alarm processing and suppress excessive alarms as compared to when the alarm processing is performed before encouraging emotional health, since the alarm processing is performed in accordance with the category and the degree of this strong emotion.

### 5. Other Embodiments

The above-described embodiment is merely one embodiment according to the present invention and can be freely modified and applied without departing from the gist of the present invention.

For example, a case is exemplified in which the vehicle control apparatus 1 acquires the driving information D1a and the traffic environment information D1c in step S13a shown in FIG. 3 and predicts future driving error behavior via the second driving error behavior detection unit 24b and the first driving error behavior prediction unit 24a in step S14a, but the present invention is not limited thereto. For example, the vehicle control apparatus 1 may acquire the driving information D1a in step S13a and detect (predict) future driving error behavior via the second driving error behavior detection unit 24b in step S14a.

A configuration realized by software, a configuration realized by hardware, or a configuration realized by combining software and hardware may be widely applied to the configuration of each unit shown in FIG. 3. A portion of configurations of the vehicle control apparatus 1, the emotion estimation apparatus, and the driving prediction apparatus described above may be provided in a system external to the target vehicle, such as a server on a communication network. In this case, information acquired in the target vehicle is transmitted to the external system, at least a portion of the processing performed by the vehicle control apparatus 1, the emotion estimation apparatus and the driving prediction apparatus is executed by the external system, output data based on a processing result is transmitted to the target vehicle, and various control in the target vehicle is realized.

The way of dividing processing units in the flowchart, the processing order thereof, and the like are not limited to the illustrated example, and may be changed as appropriate.

A case has been described in which the program 32 for realizing the vehicle control method, the emotion estimation method, and the driving prediction method according to the present invention is recorded on the vehicle control apparatus 1, but the program 32 may be acquired from an external device via communication. The program 32 may be recorded on an appropriate recording medium so as to be readable by a computer. A magneto-optical recording medium or a semiconductor memory device can be used for the recording medium.

### 6. Configurations Supported by the Above-Described Embodiments

The above embodiments are specific examples of the following configurations.

(Configuration 1) An emotion estimation apparatus includes a biological information detection unit that detects biological information of a driver of a target vehicle, an environment detection unit that detects environment information of a surrounding area of the target vehicle, and an emotion estimation unit that estimates a strong emotion of the driver based on the biological information and the environment information.

According to this configuration, quick and appropriate estimation of the strong emotion influencing the driving error behavior of the driver based on the biological information and the traffic environment information is facilitated.

(Configuration 2) The emotion estimation apparatus according to configuration 1, wherein the emotion estimation unit estimates the strong emotion using the environment information, based on an environment-emotion table indicating a relationship between an environment surrounding a vehicle and the strong emotion.

According to this configuration, it is possible to efficiently identify the strong emotion from the environment surrounding the vehicle.

(Configuration 3) The emotion estimation apparatus according to configuration 1 or 2, wherein the environment information includes at least one of road conditions, traffic density, time of day, region, and the presence or absence of a passenger.

According to this configuration, it is possible to estimate the strong emotion influenced by at least one of the road conditions, the traffic density, the time of day, the region, and the presence or absence of a passenger.

(Configuration 4) The emotion estimation apparatus according to any one of configurations 1 to 3, wherein the emotion estimation unit estimates the strong emotion using the biological information of the driver, based on a biology-emotion table indicating a relationship between the biological information of the driver and the strong emotion.

According to this configuration, it is possible to efficiently identify the strong emotion using the biological information.

(Configuration 5) The emotion estimation apparatus according to any one of configurations 1 to 4, wherein the biological information includes information that allows identification of each of an expression and a gesture of the driver.

According to this configuration, it is possible to efficiently identify the strong emotion from the expression and the gesture.

(Configuration 6) The emotion estimation apparatus according to any one of configurations 1 to 5, wherein the emotion estimation unit estimates the strong emotion, by using statistical data that allows identification of a relationship between an environment surrounding the target vehicle and the strong emotion, and driving error behavior detected from driving information of the driver in the target vehicle.

According to this configuration, the estimation accuracy for the strong emotion is improved.

(Configuration 7) An emotion estimation method executed by a computer includes a biological information detection step of detecting biological information of a driver of a target vehicle, an environment detection step of detecting environment information of a surrounding area of the target vehicle, and an emotion estimation step of estimating a strong emotion of the driver based on the biological information and the environment information.

According to this method, quick and appropriate estimation of the strong emotion influencing the driving error behavior of the driver based on the biological information and the traffic environment information is facilitated.

### REFERENCE SIGNS LIST

1 vehicle control apparatus (emotion estimation apparatus, driving prediction apparatus)
2 vehicle
3 first sensor
4 second sensor
5 third sensor
6 fourth sensor
7 vehicle drive unit
8 steering unit
9 braking unit
10 notification unit
21 processor
22 environment detection unit
22a driving information detection unit
22b biological information detection unit
22c traffic environment detection unit
23 emotion estimation unit
23a first emotion estimation unit
23b second emotion estimation unit
24 driving error behavior prediction unit
24a first driving error behavior prediction unit
24b second driving error behavior detection unit (driving error behavior detection unit)
25 driving influence degree prediction unit
25a driving risk prediction unit
26 risk suppression unit
26a control content determination unit
26b vehicle control unit
26c notification control unit
31 memory
32 program
33 map data
34 statistical database
35 position database

1. An emotion estimation apparatus, comprising:
a processor, wherein the processor:
detects biological information of a driver of a target vehicle;
detects environment information of a surrounding area of the target vehicle, the environment information including a region where the target vehicle is located, road conditions, how the driver grips a steering wheel, time information indicating a time of day such as daytime or nighttime, in-vehicle environment information indicating a presence or absence of a passenger, and weather information; and
estimates a degree of a strong emotion of the driver, the strong emotion is an emotion that influences thoughts or behavior of the driver, based on the biological information and the environment information,
wherein the processor estimates the degree of the strong emotion by calculating a sum of odds ratios for each item of the environment information based on an environment-emotion table indicating a relationship between the environment information and the odds ratio indicating how much higher a probability is of the driver experiencing the strong emotion that is the emotion that influences the thoughts or behavior of the driver as compared to an ideal condition for each item of the environment information, and
wherein the processor controls at least one of a drive unit, a steering unit, and a braking unit of the target vehicle in accordance with the estimated degree of the strong emotion.

2. (canceled)

3. (canceled)

4. The emotion estimation apparatus according to claims 1, wherein the processor estimates the degree of the strong emotion using the biological information of the driver, based on a biology-emotion table indicating a relationship between the biological information of the driver and the degree of the strong emotion.

5. The emotion estimation apparatus according to claim 1, wherein the biological information includes information that allows identification of each of an expression and a gesture of the driver.

6. The emotion estimation apparatus according to claim 1, wherein the processor estimates the degree of the strong emotion, by using statistical data that allows identification of a relationship between an environment surrounding the target vehicle and the degree of the strong emotion, and driving error behavior detected from driving information of the driver in the target vehicle.

7. An emotion estimation method executed by a computer, the emotion estimation method comprising:
a biological information detection step of detecting biological information of a driver of a target vehicle;
an environment detection step of detecting environment information of a surrounding area of the target vehicle, the environment information including a region where the target vehicle is located, road conditions, how the driver grips a steering wheel, time information indicating a time of day such as daytime or nighttime, in-vehicle environment information indicating a presence or absence of a passenger, and weather information;
an emotion estimation step of estimating a degree of a strong emotion of the driver, the strong emotion is an emotion that influences thoughts or behavior of the driver, based on the biological information and the environment information; and
a control step of controlling at least one of a drive unit, a steering unit, and a braking unit of the target vehicle in accordance with the estimated degree of the strong emotion, wherein
in the emotion estimation step, the degree of the strong emotion is estimated by calculating a sum of odds ratios for each item of the environment information based on an environment-emotion table indicating a relationship between the environment information and the odds ratio indicating how much higher a probability is of the driver experiencing the strong emotion that is the emotion that influences the thoughts or behavior of the driver as compared to an ideal condition for each item of the environment information.
